(19) United States
(12) Patent Application Publication
(10) Pub. No.: US 2004/0180966 A1
(43) Pub. Date: Sep. 16, 2004

(54) Enantiomers of N-desmethyl venlafaxine

(75) Inventors: John P. Yardley, King of Prussia, PA (US); Andre A. Asselin, Mahwah, NJ (US)

Correspondence Address: WYETH, PATENT LAW GROUP, FIVE GIRALDA FARMS, MADISON, NJ, 07940, US

(73) Assignee: Wyeth, Madison, NJ

(21) Appl. No.: 10/812,115

(22) Filed: Mar. 29, 2004

Related U.S. Application Data
(63) Continuation of application No. 10/419,646, filed on Apr. 21, 2003, now abandoned.
Application No. 10/419,646 is a continuation of application No. 10/189,215, filed on Jul. 3, 2002, now abandoned.
Application No. 10/189,215 is a continuation of application No. 09/988,690, filed on Nov. 20, 2001, now abandoned.
Application No. 09/988,690 is a continuation of application No. 09/591,548, filed on Jun. 8, 2000, now abandoned.
(60) Provisional application No. 60/183,034, filed on Jun. 15, 1999.

(51) Int. Cl. A61K 31/137

(52) U.S. Cl. 514/650; 564/339

(57) ABSTRACT

The present invention provides enantiomers of N-Desmethyl venlafaxine, as well as their use in pharmaceutical compositions and medically useful treatments, particularly including central nervous system uses.

This application claims the benefit of U.S. Provisional Application No. 60/183,034, which was converted from U.S. patent application Ser. No. 09/333,207, filed Jun. 15, 1999, pursuant to a petition filed under 37 C.F.R. 1.53(c)(2)(i).

This invention provides enantiomers of N-desmethyl venlafaxine, (R/S)-1-[1-(4-methoxyphenyl)-2-(methylamino)ethyl]cyclohexanol, as well as pharmaceutical compositions and uses thereof.

### BACKGROUND OF THE INVENTION

Various patents and literature references describe the biological activities of venlafaxine, and its salts and analogs. Venlafaxine hydrochloride tablets are marketed by Wyeth-Ayerst Laboratories under the Effexor® trademark.

The absolute configuration of the (+) enantiomer of venlafaxine was established as S by a single crystal X-ray analysis of the hydrobromide salt and the anomalous dispersion technique (Yardley et al., J. Med. Chem., 1990, 33, 2899).

(R/S)-1-[2-(dimethylamino)-1-(4-methoxyphenyl)ethyl]cyclohexanol and its metabolites 1-[2-(dimethylamino)-1-(4-hydroxyphenyl)ethyl]cyclohexanol and 1-[1-(4-methoxyphenyl)-2-(methylamino)ethyl]cyclohexanol are disclosed and claimed in U.S. Pat. No. 4,535,186 (Husbands et al.). U.S. Pat. No. 5,530,013 (Husbands et al.) claims the use of venlafaxine in the inducement of cognition enhancement. U.S. Pat. No. 5,506,270 (Upton et al.) claims venlafaxine's use in methods of treating hypothalamic amenorrhea in non-depressed women.

U.S. Pat. Nos. 5,788,986 (Dodman) and 5,554,383 (Dodman) teaches and claims the use of serotonin reuptake inhibitors in modifying the behavior of dogs.

### SUMMARY OF THE INVENTION

This invention provides pharmaceutically active enantiomers of the venlafaxine metabolite N-Desmethyl venlafaxine, particularly the S and R enantiomers of N-Desmethyl venlafaxine, having the respective general structures:

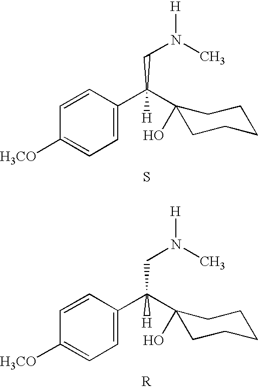

Particularly, this invention provides compositions of matter of both the R and S enantiomers substantially free of each other, as well as pharmaceutical compositions comprising each enantiomer substantially free of the other.

These enantiomers and their pharmaceutically useful salts and hydrates are useful for the biological and pharmacological activities for which venlafaxine and its salts are known in the art. The enantiomer may be used in treating or inhibiting central nervous system disorders, including depression, panic disorder, post-traumatic stress disorder, late luteal phase dysphoric disorder (also known as pre-menstrual syndrome), attention deficit disorder, with and without hyperactivity, generalized anxiety disorder, bulimia nervosa, Gilles de la Tourette Syndrome, Shy Drager Syndrome vasomotor flushing, drug and alcohol addiction, sexual dsifunction (including premature ejaculation), borderline personality disorder, chronic fatique syndrome, fibromyalgia, urinary incontinence and others. These compounds are also useful in the inducement of cognition enhancement and in regimens for cessation of smoking or other tobacco uses.

Racemic N-desmethylvenlafaxine can be produced as described in Example 12 of U.S. Pat. No. 4,535,186 (Husbands et al.) the entirety of which is incorporated herein by reference. It will be understood that the enantiomers may be separated from each other by standard resolution techniques known in the art. An example of such resolution techniques is that described by Yardley et al. for resolution of 1-[2(Dimethylamino)-1-(4-methoxyphenyl)ethyl]cyclohexanol in J. Med. Chem, Vol. 33, No. 10, at page 2904.

Pharmaceutical compositions and formulations containing the enantiomers described herein can be produced in the same fashion and containing the same dosages as those described in the art for venlafaxine hydrochloride. The pharmaceutical formulations or compositions of this invention include those having as an active ingredient the R enantiomer of N-Desmethyl venlafaxine substantially free of S enantiomer N-Desmethyl venlafaxine. This invention also includes formulations in which an active ingredient is the S enantiomer of N-Desmethyl venlafaxine substantially free of the R enantiomer of N-Desmethyl venlafaxine. Each of these formulations also comprises one or more pharmaceutically useful excipients, carriers or adjuvants.

Formulations of the present invention may be produced using the S or R enantiomer of N-Desmethyl venlafaxine, or a pharmaceutically acceptable salt or salt hydrate thereof, in the same fashion as described for venlafaxine formulations in U.S. Pat. Nos. 5,530,013 (Husbands et al.) and 5,506,270 (Upton et al.), both of which are incorporated herein by reference.

Preferred oral extended release formulations of this invention are comprised of the active enantiomer in admixture with microcrystalline cellulose and hydroxypropylmethylcellulose. Formed as beads or spheroids, the drug containing formulation is coated with a mixture of ethyl cellulose and hydroxypropylmethyl cellulose to provide the desired level of coating, generally from about two to about twelve percent on a weight/weight basis of final product or more preferably from about five to about ten percent (w/w), with best results obtained at from about 6 to about 8 percent (w/w). More specifically, the extended release spheroid formulations of this invention comprise from about 30 to 40 percent of an enantiomer of N-desmethyl venlafaxine, from about 50 to about 70 percent microcrystalline cellulose, NF, from about 0.25 to about 1 percent hydroxypropylmethylcellulose, USP, and from about 5 to about 10 percent film coating, all on a weight/weight basis. And preferably, the spheroid formulations contain about 35 percent active ingredient, about 55 to 60 percent microcrystalline cellulose NF (Avicel® PH101), about one half percent hydroxypropyl methylcellulose 2208 USP (K3, Dow, which has a viscosity of 3 cps for 2% aqueous solutions, a methoxy content of 19-24% and a hydroxypropoxy content of 4-13%), and from about 6 to 8 percent film coating.

The film coating is comprised of 80 to 90 percent of ethyl cellulose, NF and 10 to 20 percent hydroxypropyl methylcellulose (2910), USP on a weight/weight basis. Preferably the ethyl cellulose has a ethoxy content of 44.0-51% and a viscosity of 50 cps for a 5% aqueous solution and the hydroxypropylmethylcellulose is USP 2910 having a viscosity of 6 cps at 2% aqueous solution with a methoxy content of 28-30% and a hydroxypropoxy content of 7-12%. The ethyl cellulose used herein is Aqualon HG 2834.

Other equivalents of the hydroxypropylmethylcelluloses 2208 and 2910 USP and ethyl cellulose, NF, having the same chemical and physical characteristics as the proprietary products named above may be substituted in the formulation without changing the inventive concept. Important characteristics of suitable hydroxypropylmethylcelluloses include a low viscosity, preferably less than 10 cps and more preferably 2-5 cps, and a gel temperature above that of the temperature of the extrudate during extrusion. As explained below, these and other characteristics which enable the extrudate to remain moist and soft (pliable) are preferred for the hydroxypropylmethylcellulose. In the examples below, the extrudate temperature was generally 50-55° C.

Specific examples of extended release compositions of this invention include the following.

### FORMULATION EXAMPLE 1

A mixture of 44.8 parts (88.4% free base) of an enantiomer of N-desmethyl venlafaxine or a salt or hydrate thereof, such as the fumarate hydrate salt, 74.6 parts of the microcrystalline cellulose, NF, and 0.60 parts of hydroxypropylmethyl cellulose 2208, USP, can be blended with the addition of 41.0 parts water. The plastic mass of material is then extruded, spheronized and dried to provide uncoated drug containing spheroids.

Stir 38.25 parts of ethyl cellulose, NF, HG2834 and 6.75 parts of hydroxypropyl methylcellulose 2910, USP in a 1:1 v/v mixture of methylene chloride and anhydrous methanol until solution of the film coating material is complete.

To a fluidized bed of the uncoated spheroids apply 0.667 parts of coating solution per part of uncoated spheroids to obtain extended release, film coated spheroids having a coating level of 3%.

The spheroids can then be sieved to retain the coated spheroids of a particle size between 0.85 mm to 1.76 mm diameter. These selected film coated spheroids are filled into hard gelatin capsules conventionally.

### FORMULATION EXAMPLE 2

Same as for Example 1 except that 1.11 parts of the film coating solution per part of uncoated spheroids is applied to obtain a coating level of 5%.

### FORMULATION EXAMPLE 3

Same as for Example 1 except that 1.33 parts of the film coating solution is applied to 1 part of uncoated spheroids to obtain a coating level of 6%.

### FORMULATION EXAMPLE 4

Same as for Example 1 except that 1.55 parts of the film coating solution is applied to 1 part of uncoated spheroids to obtain a coating level of 7%.

One preferred extended release formulation of this invention comprises those of the active ingredient in spheroids comprised of microcrystalline cellulose and, optionally, hydroxypropylmethylcellulose coated with a mixture of ethyl cellulose and hydroxypropyl methyl cellulose. Preferably, the spheroids are comprised of about 30% to 40% venlafaxine hydrochloride by weight, about 50% to about 70% microcrystalline cellulose, NF, by weight, and from about 0.25% to about 1% by weight of hydroxypropylmethylcellulose, USP, and coated with from about 2% to about 12% of total weight of film coating comprised of from about 80% to about 90% by weight of film coating of ethyl cellulose, NF, and from about 10% to about 20% by weight of film coating of hydroxypropylmethylcellulose, USP.

A specific extended release formulation according to the paragraph above is wherein the spheroids are composed of about 37% by weight of venlafaxine hydrochloride, about 0.5% by weight of hydroxypropylmethylcellulose 2208, and about 62% by weight of microcrystalline cellulose. Another set of preferred compositions of this type are those wherein the film coating is comprised of ethyl cellulose (4.81% of total weight) and hydroxypropylmethylcellulose (0.85% of total weight). In another such composition the film coating comprises 6-8% by weight of total weight, such as a film coating comprised of ethyl cellulose (2.48% of total weight) and hydroxypropylmethylcellulose (0.437% of total weight).

Yet another composition according to this invention are those wherein the film coating composition is comprised of ethyl cellulose having a 44.0-51.0% content of ethoxy groups and hydroxypropylmethylcellulose having a methoxy content of 28.0-30.0% and a hydroxypropoxy group content of 7.0-12.0%. Film coating compositions of this type may be comprised of about 85% by total weight of film coating of ethyl cellulose having a 44.0-51.0% content of ethoxy groups, and about 15% by total weight of film coating of hydroxypropylmethylcellulose having a methoxy content of 28.0-30.0% and a hydroxypropoxy group content of 7.0-12.0%. A more specific film coating composition of this sort is comprised of 85% by weight of ethyl cellulose type HG 2834 and 15% by weight of hydroxypropylmethylcellulose type 2910.

Another extended release formulation for once daily administration of this invention comprises the N-desmethyl venlafaxine enantiomer, or a salt or hydrate thereof, which comprises spheroids containing 37.3% N-desmethyl venlafaxine enantiomer, 62.17% microcrystalline cellulose and 0.5% hydroxypropylmethylcellulose type 2208, coated with a quantity of a mixture comprised of 85% ethyl cellulose type HG 2834 and 15% hydroxypropylmethylcellulose type 2910 sufficient to give coated spheroids having a dissolution profile which gives the desired release rate over a 24 hour period.

A further extended release formulation of this invention is manufactured such that the spheroids are comprised of about 6% to 40% active compound by weight, about 50% to about 940% microcrystalline cellulose, NF, by weight, and, optionally, from about 0.25% to about 1% by weight of hydroxypropylmethylcellulose, USP, and coated with from about 2% to about 12% of total weight of film coating comprised of from about 80% to about 90% by weight of film coating of ethyl cellulose, NF, and from about 10% to about 20% by weight of film coating of hydroxypropylmethylcellulose, USP. A preferred subset of these extended release formulations are those wherein the spheroids are composed of about 8.25% by weight of active compound, or a pharmaceutically acceptable salt or hydrate thereof, and about 91.75% by weight of microcrystalline cellulose, with a coating of from 3 to 5% by weight of the total weight. Another preferred subset or group are those formulations wherein the spheroids are composed of about 16.5% by weight of active drug agent and about 83.5% by weight of microcrystalline cellulose, with a coating of from 4 to 6% by weight of the total weight.

In other pharmaceutical compositions and formulations of this invention, the active ingredient comprises venlafaxine hydrochloride combined with the N-desmethyl enantiomer, with the non-active ingredients being those described herein or in other formulations for venlafaxine hydrochloride known in the art.

Uses of these extended release formulations may be described as a method for providing a therapeutic blood plasma concentration of active drug compound(s) over a 24 hour period with diminished incidences of nausea and emesis which comprises administering orally to a patient in need thereof, an encapsulated, extended release formulation that provides a peak blood plasma level of active agent in from about four to about eight hours, said formulation containing an enantiomer of N-desmethyl venlafaxine as the active ingredient. The methods are also useful for eliminating the troughs and peaks of drug concentration in a patients blood plasma attending the therapeutic metabolism of plural daily doses of active ingredient(s) which comprises administering orally to a patient in need thereof, an encapsulated, extended release formulation that provides a peak blood plasma level of venlafaxine in from about four to about eight hours, said formulation containing an enantiomer of N-desmethyl venlafaxine, or a salt or salt hydrate thereof, as the active ingredient.

What is claimed:

1. A composition of matter comprising (R)-1-[1-(4-methoxyphenyl)-2-(methylamino)ethyl]cyclohexanol substantially free of (S)-1-[1-(4-methoxyphenyl)-2-(methylamino)ethyl]cyclohexanol, or a pharmaceutically acceptable salt or salt hydrate thereof.

2. A composition of matter comprising (S)-1-[1-(4-methoxyphenyl)-2-(methylamino)ethyl]cyclohexanol substantially free of (R)-1-[1-(4-methoxyphenyl)-2-(methylamino)ethyl]cyclohexanol, or a pharmaceutically acceptable salt or salt hydrate thereof.

3. A pharmaceutical composition comprising one or more pharmaceutically acceptable carriers and a pharmaceutically effective amount of (R)-1-[1-(4-methoxyphenyl)-2-(methylamino)ethyl]cyclohexanol substantially free of (S)-1-[1-(4-methoxyphenyl)-2-(methylamino)ethyl]cyclohexanol, or a pharmaceutically acceptable salt or salt hydrate thereof.

4. A pharmaceutical composition comprising one or more pharmaceutically acceptable carriers and a pharmaceutically effective amount of (S)-1-[1-(4-methoxyphenyl)-2-(methylamino)ethyl]cyclohexanol substantially free of (R)-1-[1-(4-methoxyphenyl)-2-(methylamino)ethyl]cyclohexanol, or a pharmaceutically acceptable salt or salt hydrate thereof.

5. A method of treatment of depression in a mammal, the method comprising administering to a mammal in need thereof a pharmaceutically effective amount of (R)-1-[1-(4-methoxyphenyl)-2-(methylamino)ethyl]cyclohexanol substantially free of (S)-1-[1-(4-methoxyphenyl)-2-(methylamino)ethyl]cyclohexanol, or a pharmaceutically acceptable salt or salt hydrate thereof.

6. A method of treatment of depression in a mammal, the method comprising administering to a mammal in need thereof a pharmaceutically effective amount of (S)-1-[1-(4-methoxyphenyl)-2-(methylamino)ethyl]cyclohexanol substantially free of (R)-1-[1-(4-methoxyphenyl)-2-(methylamino)ethyl]cyclohexanol, or a pharmaceutically acceptable salt or salt hydrate thereof.
